(19) United States
(12) Patent Application Publication
(10) Pub. No.: US 2013/0204158 A1
(43) Pub. Date: Aug. 8, 2013

(54) METHOD OF DIAGNOSIS AND LOCATION OF A SOFT TISSUE INJURY

(71) Applicant: Mark John McDonald Rogers, Parkside (AU)

(72) Inventor: Mark John McDonald Rogers, Parkside (AU)

(21) Appl. No.: 13/700,375

(22) PCT Filed: May 23, 2011
(86) PCT No.: PCT/AU11/00609

§ 371 Date: Feb. 14, 2013

(30) Foreign Application Priority Data
May 27, 2010 (AU) 2010902320
Dec. 6, 2010 (AU) 2010905350

(51) Int. Cl. A61B 5/00; A61B 5/01

(52) U.S. Cl. CPC A61B 5/4824; A61B 5/015; A61B 5/0082
USPC 600/549; 600/300

(57) ABSTRACT

A method of diagnosing, and determining the position of a microscopic or a macroscopic soft tissue injury or soft tissue stress fracture in a patient. The method includes the steps of determining a pain area on the skin of the patient, applying electromagnetic energy or radiation in a selected portion or range of the visible or infrared spectrums, to parts of the body's surface that correspond with the pain area, obtaining feedback from the patient to determine the sensations that the patient experiences as a result of visible or infrared energy being applied to the tissue at a specific region of pain area, and establishing the site of the microscopic or macroscopic soft tissue injury at the specific region where the sensations are greatest. The visible or infrared energy can be applied using a laser probe. The probe can operated at a selected wavelength or a set of wavelengths in the range of 400 nm to 10,000 ran. The soft tissue injuries to which the present invention may be applicable include injuries that result in symptoms including lower back pain, neck pain, migraines, Type 2 diabetes, sciatica, tinnitus, carpal tunnel syndrome, chronic pain syndrome and fibromyalgia.

### FIELD OF THE INVENTION

The present invention relates to a method of medical diagnosis and more particularly to a method of diagnosis and location of a soft tissue injury.

### BACKGROUND

Soft tissue injuries are identified as a major source of pain and disability and occur across a wide section of the community.

Soft tissue injuries arise generally as a result of damage to muscles, nerves, connective tissues, fascia, joint capsules, periosteum etc as a result of excessive force/stress in a given moment, or repetitive strain placed upon these tissues over an extended period of time. As such, soft tissue injuries are very common in the workplace. Additionally, soft tissue injuries that occur as a result of trauma may not be immediately obvious to the individual at the time of the trauma but may become apparent at some point in the future.

A soft tissue injury can be considered to be a fracture because it is the local separation of a body into two, or more pieces under the action of stress. Hence damage to soft tissue can be referred to by either of the terms soft tissue stress fracture or soft tissue injury and can be used interchangeably.

A method to identify soft tissue damage is with the use of Magnetic Resonance Imaging (MRI). Such equipment requires detailed understanding of the symptoms of the injured person, his/her case history, and then, based on that information, very precise and localised use of the equipment to observe a microscopic injury. The equipment used for this form of imagery is very expensive and therefore cannot be used day-to-day by general practitioners and as such MRI is not considered to be a useful tool for general diagnosis of soft tissue injuries.

Inflammation of soft tissue is a result of a complex cascade of events that includes changes to concentration of various chemical components within the body, such as histamines, prostaglandins, cytokines etc along with inflammatory cells such as leukocytes, fibroblasts and macrophages. The inflammatory response results, physiologically, in an increase in inflammatory hormones and/or nerve chemicals at the site of injury, swelling, hypersensitivity, neuritis, fasciculation, involuntary muscle contraction, heat, reduced blood flow, and critically, a reduced ability of the lymphatic system to drain interstitial fluid (lymphoedema). All of this causes a vicious cycle of pain for the individual.

It is a commonly-held belief that infrared thermography detects differences in heat, and therefore inflammation. However, infrared imaging is actually detecting a selected range of infrared wavelengths (photons with wavelengths in the range of 700 nm to 2000 nm, or in the range of 810 nm to 820 nm, as two possible examples). The hotter a body of matter is, the more infrared intensity it emits in this infrared spectrum range. Therefore, within a specified wavelength range, the amount of heat corresponds to the infrared intensity within that wavelength range. The narrower the wavelength range, the smaller the range of temperatures that the thermograph can detect. Recently, digital infrared thermographs that correspond to the narrow temperature range of metabolic heat have been demonstrated, enabling highly accurate thermographs of the surface temperature of the human body.

These methods, however, simply rely on a change of the surface temperature and are insufficient to provide a reliable diagnosis. The surface temperature is only an indirect indication of the temperature at the site of the injury, which is located inside the body of the soft tissue. There is no current method for accurately detecting the precise location and the amount of inflammation around the site a soft tissue injury. There exists, therefore, a need for an accurate, non-invasive, rapid and inexpensive method of detecting inflammation below the surface of the body.

### SUMMARY OF THE INVENTION

According to the present invention, although this should not be seen as limiting the invention in any way, there is provided a method of diagnosing, and determining the position of, a microscopic or a macroscopic soft tissue injury or soft tissue stress fracture in a patient, including the steps of:

determining on the skin of the patient a pain area;

applying electromagnetic energy or radiation, in a selected portion or range of the visible or infrared spectrums, to parts of the body's surface that correspond with the pain area;

obtaining feedback from the patient to determine the sensations that the patient experiences as a result of visible or infrared energy being applied to the tissue at a specific region of pain area; and

establishing the site of the microscopic or macroscopic soft tissue injury at the specific region where the sensations are greatest.

In this way, a primary soft tissue injury site may be determined as a result of tingling, aching, heat, or ‘pins and needles’ sensations travelling along a nerve of the patient to a site distal from the visible or infrared laser probe.

The explanation which the inventor believes explains the observed reaction to the application of visible or infrared energy, but to which the inventor does not necessarily wish to be restricted, is that the visible or infrared energy interacts with cells and proteins which are accumulated at a site of microscopic or macroscopic soft tissue injury in a body and thereby provide the observed sensations.

The inventor has observed that the energy from the visible or infrared spectrums is not absorbed at sites where there is no inflammation but it is absorbed at sites where there is inflammation. This has been observed from both patient feedback of sensations at the site of inflammation and also indicative data from monitoring the change in the digital infrared thermographs of the surface of the body, near the site of the soft tissue injury.

It is believed that the photons from the visible or infrared spectrums interact with the inflammatory cells and proteins along a nerve fibre connected to the soft tissue injury thereby providing the observed sensations.

In one embodiment of the invention the step of determining on the skin of the patient a pain area comprises the step of observing surface temperature on the skin of the patient with the highest surface temperature indicating a pain area.

In an alternative embodiment of the invention the step of determining on the skin of the patient a pain area comprises the steps of:

obtaining a thermographic image of the pain area of the patient to enable visualization of variation in surface temperature of the pain area;

reviewing the thermographic image to determine the point or points of greatest surface temperature; and

applying the electromagnetic energy or radiation to the point or points of greatest surface temperature.

Preferably the application of the visible or infrared energy at any one point is for no longer than two or three minutes.

Preferably the visible or infrared energy is applied at a selected wavelength or a set of wavelengths in the visible, near-infrared and infra-red wavelength spectrums.

Preferably the visible or infrared energy is applied using a laser probe and which is applied via direct contact of the laser probe with the patient's skin, delivered via a fibre optic delivery system from the laser probe to the patient's skin, or delivered by pointing the beam from the laser probe through the air to the patients skin. Alternatively the visible or infrared energy is being applied using a probe or optical emitter device other than a laser device, such as a Light Emitting Diode (LED), a light bulb or similar optical emitter.

Preferably the probe is operated at a selected wavelength or a set of wavelengths in the range of 400 nm to 10,000 nm, which corresponds to wavelengths in the visible, near-infrared and infra-red wavelength spectrums.

The step of obtaining feedback from the patient can comprise establishing dialogue with the patient to understand the sensations that they experience as a result of visible or infrared energy being applied to the tissue at the point or points.

Alternatively the step of obtaining feedback from the patient comprises using a feedback device, such as a switch, a lever or a rotating variable knob as examples, that the patient can input the presence or not of sensation and the amount of sensation, in real time.

Preferably the invention can further including an initial step of obtaining a case-history of the patient to determine possible areas of injury and pain areas. This additional step may or may not be beneficial to the diagnostic process, depending on the accuracy and success of previous diagnoses and treatments.

The method of the present invention can be used to diagnoses soft-tissue injuries that result in symptoms such as lower back pain, neck pain, migraines, Type 2 diabetes, sciatica, tinnitus, carpal tunnel syndrome, chronic pain syndrome, fibromyalgia or be used to diagnoses soft-tissue injuries that result in other symptoms that are either not known at this time or not described above. Hence the method of the present invention may be used to diagnose and determine the site of an injury which is not apparent as observed by other techniques and which can be the original source of their pain.

Once a diagnosis has been obtained by determining the site of the microscopic or macroscopic soft tissue injury treatment can be applied using a visible or infrared laser probe.

In preference, for treatment of a soft tissue injury there may be 2×300 mW 830 nm infrared lasers used for periods greater than five minutes and less than 60 minutes per treatment. In preference, the time of application of visible or infrared energy to an injury site is greater than five minutes and less than eight minutes per treatment but no more than two to three minutes at any one time.

### BRIEF DESCRIPTION OF THE DRAWINGS

By way of example, an employment of the invention is described more fully hereinafter with reference to the accompanying drawings, in which:

FIG. 1 shows a schematic overview of the present invention.

### DETAILED DESCRIPTION OF THE INVENTION

In a preferred embodiment of the invention as a first step an infrared thermographic image (thermogram) is taken to observe the patient's dermatomal neurophysiology of pain areas. An interpretation of the thermogram can be given to the patient regarding their general problem area but then further diagnosis is necessary according to the present invention to refine in more detail the site of the injury. This is done as discussed above by application of visible or infrared energy using a laser probe and obtaining feedback as to sensations felt by a patient.

Usually, but not always, the laser will take five to eight minutes at the start of a diagnosis session to generate enough energy to produce any perceivable sensation in the patient. Visible or infrared laser energy is applied to the site of pain and/or hotspots indicated on the thermogram for a period of no less than one minute and no more than three minutes.

The patient is advised that it is very important to communicate the sensations that they feel in their body during therapy. Sensations of heat and pain are best communicated using a feedback arrangement based upon a scale of zero to 10 with zero being no pain/heat (cold)’ and 10 being ‘too painful/hot, please move the probe’. Other sensations, such as tingling, ‘pins and needles’, dull aches, bubbling, numbness, “ants crawling under my skin” and many more may be communicated without a scale.

When heat or other sensation is experienced by the patient, at the location of the laser, inflammation is being detected.

When radiating sensations are experienced by the patient to distal parts of the body, such as heat, tingling, aching, pins and needles etc, soft tissue stress fracture have been detected.

According to the interpretation of the inventor, the patient is experiencing sensations of the inflamed neuron(s) within ruptured collagen fibres. Applying an amount of infrared energy to the site of the injury stimulates or excites nerve chemicals and/or inflammatory proteins, such as histamines, prostaglandins, substance P, kinins, bradykinins etc along the neuron localised from the injury site. The injury is a source, or cause, of inflammation being present in the region.

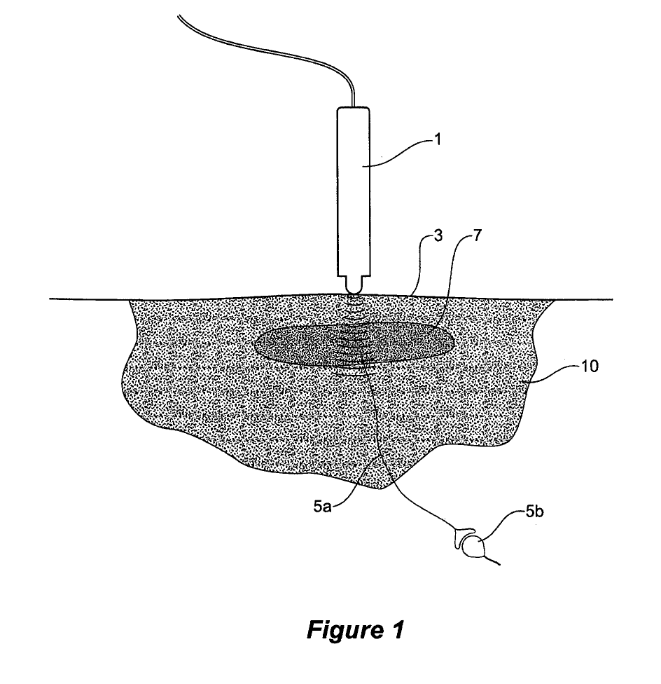

FIG. 1 shows an overview of this process in which visible or infrared energy from a probe source 1 is applied to an inflammatory site 10 of an area of the patient's body 3. This site 10 is identified by obtaining a obtaining a thermographic image of a pain area of the patient to enable visualization of variation in surface temperature of the pain area. The visible or infrared energy, at the frequencies used, is able to penetrate the body and can come into contact with a soft tissue stress fracture 7 and an associated nerve fibre 5a connecting to nerve fibre 5b to a distal location of the body. The visible or infrared energy travels along nerve fibre 5a exciting the inflammation proteins within the nerve itself. As infrared energy travels along the length of the nerve fibre 5a to a neuron 5b more of the inflammatory proteins are excited causing referred sensations as discussed above, enabling a diagnosis to be made as to the site where the laser energy meets the soft tissue injury (stress fracture) 7.

When the patient experiences referred sensation (i.e. sensations at a location distant from the probe caused by visible or infrared energy), the laser energy is travelling along the neuron(s) and is having a far-reaching effect on the patient.

The visible or infrared laser may be moved every one to three minutes to a nearby location. The probe is required to stay in one location for at least one minute to assess whether any perceivable sensations are occurring (as some sensations build over time) and not more than three minutes to avoid bioinhibition of healing.

The nearby location may be right next to the previous spot or in a completely new area depending on what areas of the body have been treated already as well as the case-history of the patient and the results of the thermographic image.

After moving the laser from an area of the patient's body that created significant sensations, the therapist will move to a new location for a period of time to avoid bio-inhibition of the injury before coming back to the area of significance.

Avoiding bioinhibition can be a fine line, but if the therapist sticks to the general guideline of not treating an injury for greater than eight minutes per therapy session, the results will be positive. This is imperative to avoid the possible anti-therapeutic effects of electromagnetic radiation. Various modifications may be made in details of design and construction and process steps, parameters of operation etc without departing from the scope and ambit of the invention.

1. A method of diagnosing, and determining the position of, a microscopic or a macroscopic soft tissue injury or soft tissue stress fracture in a patient, including the steps of:
determining a pain area on the skin of the patient;
applying electromagnetic energy or radiation, in a selected portion or range of the visible or infrared spectrums, to parts of the body's surface that correspond with the pain area;
obtaining feedback from the patient to determine the sensations that the patient experiences as a result of visible or infrared energy being applied to the tissue at a specific region of pain area; and
establishing the site of the microscopic or macroscopic soft tissue injury at the specific region where the sensations are greatest.

2. A method as in claim 1 wherein the step of determining on the skin of the patient a pain area comprises the step of observing surface temperature on the skin of the patient with the highest surface temperature indicating a pain area.

3. A method as in claim 1 wherein the step of determining on the skin of the patient a pain area comprises the steps of:
obtaining a thermographic image of the pain area of the patient to enable visualization of variation in surface temperature of the pain area;
reviewing the thermographic image to determine the point or points of greatest surface temperature; and
applying the electromagnetic energy or radiation to the point or points of greatest surface temperature.

4. A method as in any one previous claim wherein the application of the visible or infrared energy is at any one point is for no longer than two or three minutes.

5. A method as in any one previous claim wherein the visible or infrared energy is applied at a selected wavelength or a set of wavelengths in the visible, near-infrared and infra-red wavelength spectrums.

6. A method as in any one previous claim wherein the visible or infrared energy is be applied using a laser probe.

7. A method as in any one previous claim wherein the visible or infrared energy is applied using a laser probe and which is applied via direct contact of the laser probe with the patient's skin, delivered via a fibre optic delivery system from the laser probe to the patient's skin, or delivered by pointing the beam from the laser probe through the air to the patients skin.

8. A method as in any one previous claim wherein the visible or infrared energy is being applied using a probe or optical emitter device other than a laser device, such as a Light Emitting Diode (LED), a light bulb or similar optical emitter.

9. A method as in claim 6 wherein the probe is operated at a selected wavelength or a set of wavelengths in the range of 400 nm to 10,000 nm, which corresponds to wavelengths in the visible, near-infrared and infra-red wavelength spectrums.

10. A method as in any one previous claim wherein the step of obtaining feedback from the patient comprises establishing dialogue with the patient to understand the sensations that they experience as a result of visible or infrared energy being applied to the tissue at the point or points.

11. A method as in any one previous claim wherein the step of obtaining feedback from the patient comprises using a feedback device, such as a switch, a lever or a rotating variable knob as examples, that the patient can input the presence of sensation and the amount of sensation, in real time.

12. A method as in any one previous claim further including an initial step of obtaining a case-history of the patient to determine possible areas of injury and pain areas.

13. A method as in any one previous claim that is used to diagnose soft-tissue injuries that result in symptoms selected from the group comprising lower back pain, neck pain, migraines, Type 2 diabetes, sciatica, tinnitus, carpal tunnel syndrome, chronic pain syndrome and fibromyalgia.

14. A method as in any one previous claim that is used to diagnose soft-tissue injuries that result in other symptoms that are either not known at this time or not described in claim 13.
